(19) United States
(12) Patent Application Publication
(10) Pub. No.: US 2013/0324971 A1
(43) Pub. Date: Dec. 5, 2013

(54) CORPOREAL CATHETER

(71) Applicant: Radius International, Inc., Grayslake, IL (US)

(72) Inventor: David G. Quinn, Grayslake, IL (US)

(21) Appl. No.: 13/803,836

(22) Filed: Mar. 14, 2013

Related U.S. Application Data
(60) Provisional application No. 61/654,448, filed on Jun. 1, 2012.

(51) Int. Cl. A61M 25/00

(52) U.S. Cl. CPC A61M 25/0068
USPC 604/523

(57) ABSTRACT

A corporeal catheter comprising a catheter tube having a distal end to which a bolus is connected. The bolus contains a tip section and a tube connector section between which a bolus passage section is disposed. The bolus passage section has a passage therein which communicates through a radial port area with the outside of the generally tubular shaped bolus. The port area includes a port mouth which is elongated between opposed elliptical curved surfaces defining a concave arch. Opposite the concave arch port mouth, under the port is a convex stiffening arch on the bolus and axially aligned with it.

### RELATED APPLICATIONS

This application is based upon Provisional Application Ser. No. 61/654,448 filed on Jun. 1, 2012, and claims priority therefrom. Provisional Application Ser. No. 61/654,448 is incorporated herein by reference, in its entirety.

### FIELD OF INVENTION

The invention relates to corporeal catheters for use in administering fluids to body cavities, especially the stomach or intestine, or irrigating the cavities and aspirating the cavities. It relates particularly to catheters having distal ends that contain opening(s) or ports for fluid egress or ingress. Applications for these catheters also include wound drainage and the use of endotracheal tubes for pulmonary usage. The basic invention may also relate to intravenous catheters utilized for the instillation or evacuation of fluids in the circulatory system. It may also relate to urology catheters for accessing the bladder.

### BACKGROUND OF THE INVENTION

Catheters are commonly used for enteral feeding, urinary bladder drainage and irrigation, suctioning of blood and mucosa, and for other purposes in the medical treatment of humans. Exemplary catheters are illustrated and described in U.S. Pat. Nos. 4,594,074, No. 4,410,320, No. 4,390,017, No. 4,388,076, No. 4,220,542, No. 5,451,216 and No. 5,810,787. Each of these catheters employs a tube with a bolus at its distal end and an opening or port extending either axially from the tube end bolus or from its side.

In the catheters illustrated in U.S. Pat. No. 5,451,216 and U.S. Pat. No. 5,810,787, the ability to aspirate is enhanced by the use of an enlarged size of the opening or port. This size increase is achieved in the '787 patent catheter by extending the port opening to upstanding side-walls of the bolus. The enlarged port effectively encloses at least 180 degrees of the inside circumference of the passage in the bolus. This bolus passage also incorporates a floor of the passage that curves upwardly in an arc of substantial radius extending axially of the catheter tube and bolus. These bolus designs approximate the fluid flow characteristics of an open-ended tube, and fluid flow out of the port is achieved at a rate of approximately 100% of the tube outflow rate. Clinical studies show, that in both of these designs, aspiration can be achieved in 85% of attempts versus only 15% in earlier other designs.

It is recognized that 100% aspiration could be achieved by increasing the size of the bolus by either lengthening it or by decreasing the height of the substantially vertical side-wall portions bracketing the port to a level beyond 180 degrees. However, making such changes to the port result in weakening the bolus, with resultant kinking and occlusion of the bolus at the port. The hereinafter described catheter invention of the present application increases the overall effective size of a bolus port by 41% over that described in the '787 patent and achieves an aspiration rate of 100% while preventing any bolus kinking.

### SUMMARY OF INVENTION

A primary object of the present invention is to provide a corporeal catheter including an improved bolus for a catheter tube.

Another object is to provide a new and improved enteral, single lumen feeding tip that greatly reduces clogging.

Yet another object is to provide a new, enteral single lumen feeding tip bolus that increases the ability to aspirate gastric or jejunal contents at a rate of up to as much as 100% of the catheter tube flow rate.

A further object is to provide a bolus having a radial port size increase from 189 degrees of the total 360 degrees of the bolus tip outside diameter to 206 degrees of this outside diameter.

Another object is to provide a bolus having a port whose depth is increased from previous catheter bolus depth of slightly below the catheter tube lumen midpoint or radius to a depth that is slightly below 75% of the tube bolus lumen inside diameter.

Another object is to provide a bolus port that never produces restrictions of any kind that are less than the internal cross-sectional area of the catheter tube lumen.

Still another object of the invention is to provide a single lumen bolus tip having the lowest possible port sides, of height and resultant largest cross-section of the bolus port passage while, at the same time, creating a bolus tip that does not bend.

Yet another object of the present invention is embodied in a catheter bolus port which is effectively enlarged in its length without increasing the actual length of the bolus.

Still another object is to provide a catheter bolus which has an overall shape that reduces, and minimizes, both patient pain and mucosal damage during insertion, retention and tube removal.

The foregoing and other objects of the invention are embodied in an improved catheter including a bolus with an enlarged and improved port for delivering fluid to a body cavity or aspirating the cavity. The bolus includes a tubular shaped body formed of semi-rigid plastic. The body of the bolus contains a tube connection section at one end, a tip section at the other end and passage section between the connector section and the tip section. The passage section also contains a symmetrical radial passage portion adjacent the tip section that foams a port through the side of the body. This passage portion contains two identical elliptical surface portions that connect the top-most section of the symmetrical radial passage to the tube connector section and the tip section. This passage section also contains a curving floor that curves upwardly to form a flow direction passage that terminates at the midpoint of a port defining concave arch. The passage section further includes a bolus body section opposite the port. The body section connects the tip section with the connector section. Side walls are formed only in the bottom one-half of the passage section that is under the one-half of a concave radial arch that connects to the connector section. The passage, at its lowest point, has a height that is slightly lower than 75% of the inside diameter of the tube lumen, or 206 degrees. The body segment also includes a structural component protruding radially outwardly therefrom, and is effective to prevent the body segment from bending and restricting the port. This structural component is also symmetrical about its midpoint, directly aligned perpendically with the midpoint of the symmetrical radial passage forming the port opening through the side of the body, thereby further preventing kinking, bending and resultant flow restriction by acting with each other to distribute forces which effectively produce reinforcement of the bolus.

### BRIEF DESCRIPTION OF THE DRAWINGS

The invention, including its construction and method of operation, is illustrated more or less diagrammatically in the drawings, in which:

FIG. 1 is a side view showing the catheter bolus connected to the catheter tube, and showing the bolus port shape;

FIG. 2 is a top plan view of the bolus end of the catheter seen in FIG. 1, showing the port and identifying the portion of the port that is recessed behind the vertical side walls of the bolus tube connector;

FIG. 3 is a front end view of the catheter bolus, bullet tip as seen in FIG. 1;

FIG. 4 is a perspective view of the catheter seen in FIG. 1, and again shows the portion of the bolus port that is recessed behind the aforementioned vertical port walls, the recessed portion of the port comprising 50% of the length of the port (note the remaining 50% of the port having no side walls);

FIG. 5 is side elevational view of the bolus shown in FIG. 1 showing cross section lines for FIGS. 6-13 which follow;

FIG. 6 is a sectional view taken along line 6-6 of FIG. 5 showing the cross-section of the catheter;

FIG. 7 is a sectional view taken along line 7-7 of FIG. 5 showing the tube and bolus connecting portion of the bolus;

FIG. 8 is a sectional view taken along line 8-8 of FIG. 5 showing the proximal initial portion of the bolus port;

FIG. 9 is a sectional view taken along line 9-9 of FIG. 5 showing the midpoint of the bolus portion that has substantially vertical side-walls;

FIG. 10 is a sectional view taken along line 10-10 of FIG. 5 showing the lowest point of the port where the port transitions from a port with side-walls;

FIG. 11 is a sectional view taken along line 11-11 of FIG. 5 showing the shape of the bolus as it ascends;

FIG. 12 is a sectional view taken along line 12-12 of FIG. 5 showing the continuation of the ascent of the port without side-walls at the point where the ellipse that forms the top distal end of the port, and the bullet tip begins;

FIG. 13 is a sectional view taken along line 13-13 of FIG. 5 at the midpoint of the bullet tip;

FIG. 14 is a longitudinal sectional views showing the tube/tip connection and the sloping floor of the port where this sloping floor meets the mid point of the port and where the side walls end and the end of the tube;

FIG. 15 is a side elevational view of the catheter bolus shown in FIG. 1 and, also, showing the separated bottom arch and the smaller upper arch on the same vertical perpendicular axis that forms the bolus bottom and the main bolus port, as well as the two identical smaller ellipses and the segments that form the top proximal and distal ends of the bolus port shown in FIG. 1;

FIG. 16 is a sectional view taken along line 14-14 of FIG. 5 also showing a phantom cross-section of the vertical side of the bolus including its cross-section in square inches;

FIG. 17 is a cross-section similar to FIG. 16, but of the bolus in Quinn U.S. Pat. No. 5,810,787, the phantom cross-section with its size is shown as it is in FIG. 16;

FIG. 18 is a cross-section similar to FIG. 10 showing the lowest point of the bolus port at 206 degrees of the OD of the bolus and the radial axis of the tube lumen;

FIG. 19 is a cross section of the bolus of the tip covered by U.S. Pat. No. 5,810,787 where the lowest point of the bolus is slightly below the radius of the bolus covering 186 degrees of the bolus;

FIG. 20 is a cross-section of the bolus covered by Quinn expired patent U.S. Pat. No. 4,594,074 where the vertical port side-walls are lowered to the level of the top of the internal lumen of the bolus;

FIG. 21 is a cross-section of a conventional punched side hole port in a bolus or a tube;

FIG. 22 is a perspective view of the bolus port's “phantom” shape showing its “wrapped around” configuration;

FIG. 23 is a “flattened” version of the shape shown in FIG. 22; this version showing the actual cross-sectional area of the bolus of the invention as it surrounds 206 degrees of the bolus;

FIG. 24 is a “flattened” version of the port size of U.S. Pat. No. 5,810,787 that surround 186 degrees of the bolus (it is approximately 29% smaller than the port shown in FIG. 23);

FIG. 25 is a flattened version of the port size illustration of U.S. Pat. No. 4,594,074 that covers only 115 degrees of the bolus port, a cross-sectional area that is less than half of that of the bolus port in the present invention;

FIG. 26 is a flattened version of a conventional side hole port.

FIG. 27 is a side elevational view of the catheter of FIG. 1 showing the relation of an adjacent jejunum wall;

FIG. 28 is a side elevational view of another version of the invention wherein the bolus is formed on the actual tube by “over-molding” to the tube, and the tube is skived to form approximately 50% of the actual port (over-molding generally is described in Quinn U.S. Pat. No. 7,988,658);

FIG. 29 is a sectional view of FIG. 28 and shows the formation of the bolus with the floor of the port passage curving to meet the midpoint of the port, the bottom convex arch forming the bottom of the bolus and the top smaller concave arch forming the vertical side-walls of the port are aligned again on same perpendicular axis;

FIG. 30 is a sectional view of FIG. 28 showing the skived tube.

### DESCRIPTION OF THE PREFERRED EMBODIMENTS

Preferred embodiments of the invention are described here in the context of a catheter for use in enteral feeding. However, features of the invention may apply equally well to all types of catheters, including Foley catheters, urethral catheters and catheters for use in such diverse applications as intravenous, pharyngeal, esophageal, recta-colonic, gastric, nasal, wound drainage and endo-bronchial procedures, as well as others.

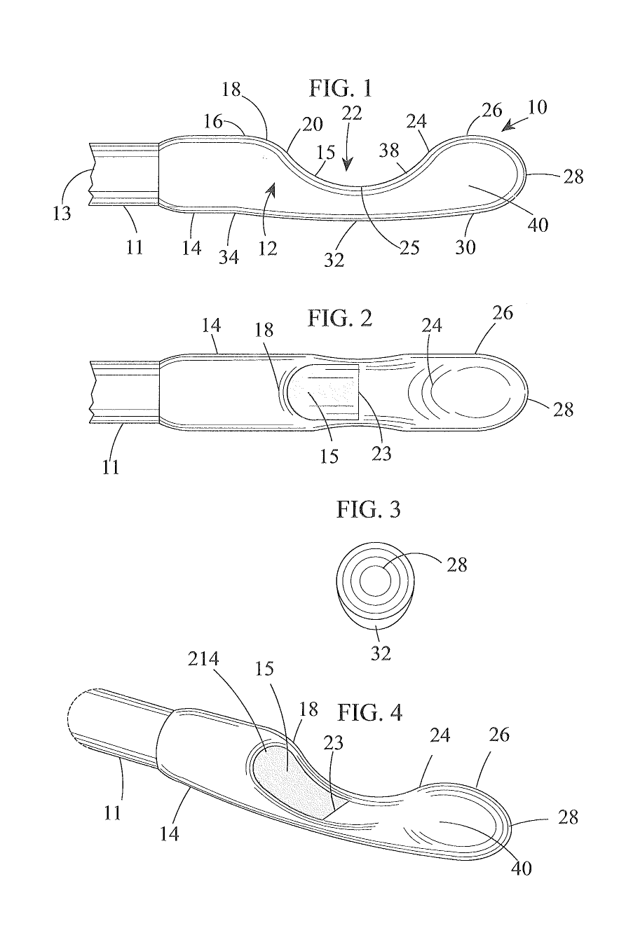

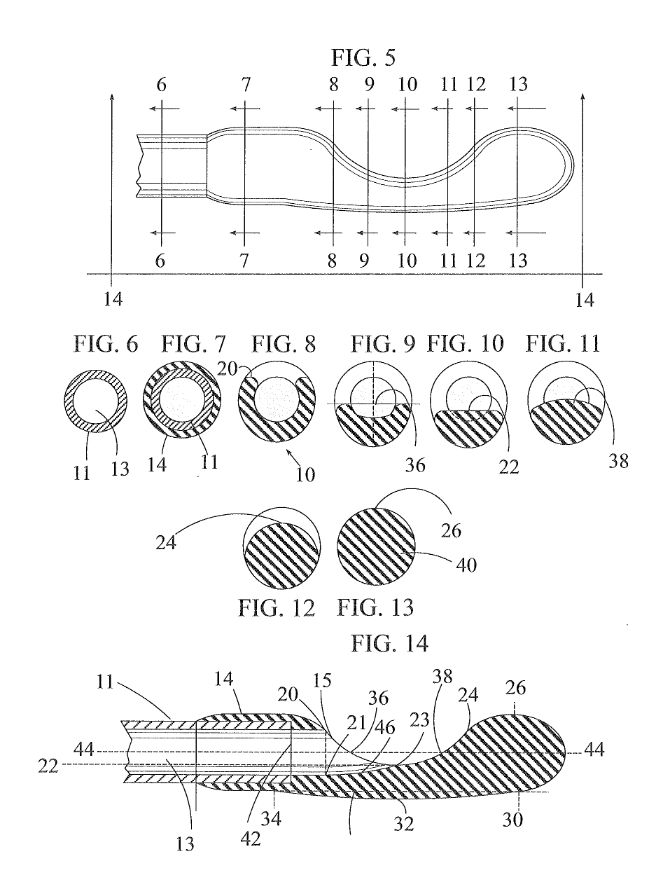

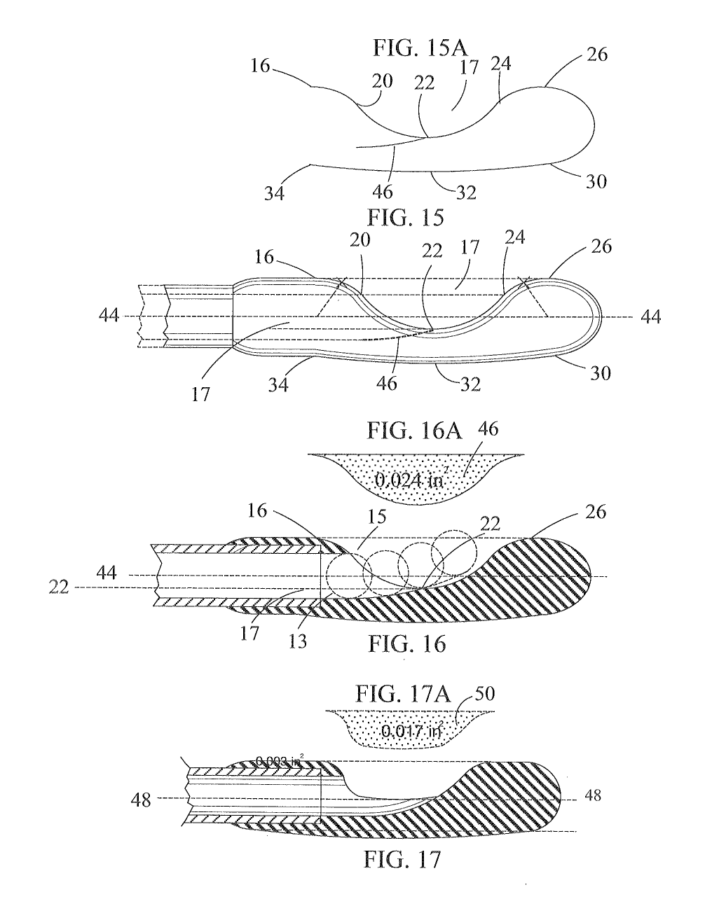

Referring now to the drawings, and particularly to FIG. 1, a catheter 10 comprising a catheter tube 11 (partially shown) and a bolus tip 12 is illustrated in side elevation. The catheter tube 11 is fabricated from a resilient, biocompatible, thermoplastic material such polyurethane. Although other plastics, including polyvinylchloride, may be used, the properties of polyurethane are such that it can be fabricated with maximum inside tube lumen diameter and minimum tube wall thickness. It also has a high resistance to highly acidic fluids frequently encountered in clinical applications. Thermo-set materials such as silicone may also be used, however.

The tube 11 used as an example is shown as a 9FR (FRENCH) tube, which has an OD (outside diameter) of 0.124 inches. The tube lumen 13 has an cylindrical ID (inside diameter) of 0.090 inches. Common FR sizes for this type of catheter tube 13 run from the smallest 3FR to tubes as large as 30FR. The bolus 12 is also fabricated from polyurethane with a hardness in the range of 80A.

Still referring to FIG. 1, the bolus 12 has an external concave arch (arcuate external protuberance) 32 extending from the tube 11 at proximal point 34 to a distal point 30 where it meets elliptical profile bullet bolus tip 40. This arch 32 extends outwardly and longitudinally of the tube 11 axis and has a radius of 1.903 inches (axially extending). At its midpoint the arch 32 extends downwardly (radially) 0.015 inches to its deepest point from the OD of the bolus tube gluing section 14. Both ends of the bolus 12 terminate at the same longitudinal level, that is, the OD of the bolus gluing section 14. This external arch 32 is important because it provides stiffness to the bolus 12 through the bolus port area 22 above. The arch 32, as shown, has a length of 0.480 inches (axially).

The bolus inflow/outflow port 15 is defined opposite the external reinforcing convex arch 32 by an inwardly extending structural concave arch 23 that begins at point 20 where it connects with ascending small ellipse 18 and forms the port area 22. Arch 23 descends from point 20 and then ascends to point 24. Point 24 is the beginning of ellipse 28 that forms the bolus bullet tip. Point 25 is the midpoint of the inner concave arch 23. Arch 23 has a radius of 0.179 inches.

Arches 23 and 32 both have their midpoint on the same imaginary line that is perpendicular to the longitudinal internal axis 44 of tube 11. This relationship is shown graphically in FIG. 15. The alignment of the two arches radii in relation to each other is very important to providing a bolus that can flex against a body cavity or mucosa, while at the same time not kinking. The convex long arch 32 provides anti-kinking protection over the full length of the upper bolus area defined by the radius of the concave upper arch 23. This lower bolus arch 32 must be stretched when kinking pressures are applied.

Now referring to both FIG. 1 and FIG. 14, the floor of the tube 11 lumen begins to rise and form a 0.524 inch radius 46 at the point 20 where the bolus port 15 becomes fully open. This radius 46 terminates at the midpoint 25 of upper arch 23. This connection between floor radius 46 and radius of the arch 23 is critical to the strengthening of arch 23. The distal one-half of arch 23 is now solid plastic. The proximal one-half of the arch 23 is trapped by solid top 40 point and the open edge of ellipse 18. Because radii 23 and 32 are aligned with each other, they provide maximum rigidness while, at the same time, providing resistance against kinking.

Now referring to FIG. 1 and FIG. 15 the top open portion of the port 15 is formed by ellipses 18 and distal ellipse 28. Distal ellipses 28 forms the leading edge of ellipse 28 that also extends to form the bullet tip. Both ellipses dimensions are X 0.107 inches and Y 0.077 inches. These ellipses connect to the main radius of the arch concave upper 23 tangentially and serve two purposes. First, they provide an effectively larger recessed port 15 than if they were radii. Second they provide a smoother transition to the bolus OD thereby providing a tissue-friendly transition during tube placement, tube removal and when in-situ in a vessel of body cavity. The proximal ellipse 18 transitions from 16 to 20 and the distal ellipse transitions from point 24 to 26. As shown graphically in FIG. 15 both ellipses have their axis from center radius 44 of the tube 11.

FIGS. 16 shows the vertical port cross-section phantom as defined by concave arch 23, ellipse 18 and ellipse 28 of the invention. Also shown are phantom tube lumens 13 exiting unrestricted through port 15.

FIG. 17 shows the vertical cross section of the bolus described in Quinn U.S. Pat. No. 5,810,787. The present bolus invention 12 has a 41% larger vertical cross sectional area than the bolus in Quinn US Pat. No. 5,810,787.

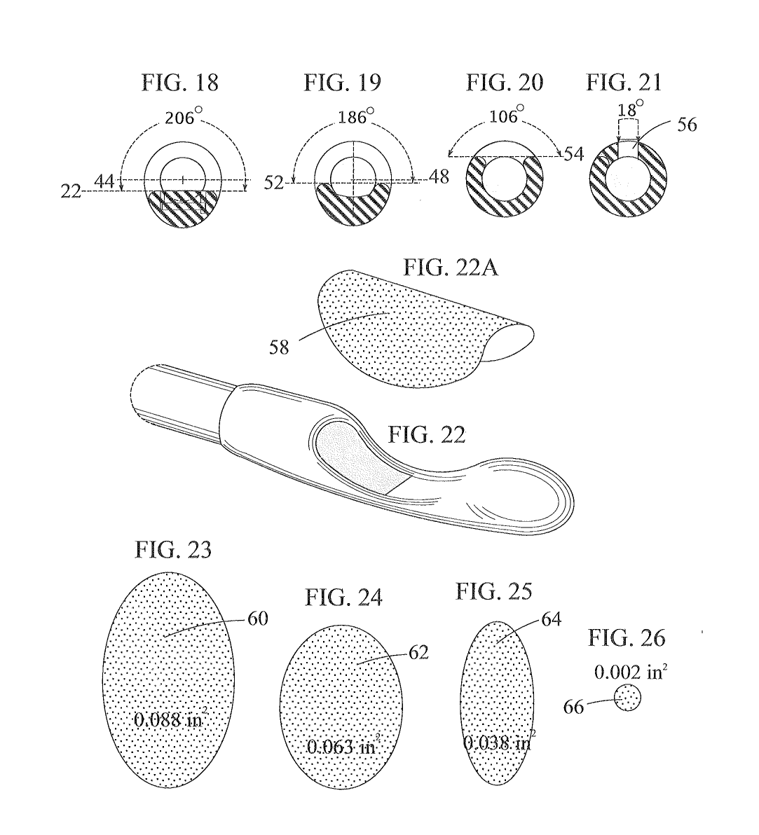

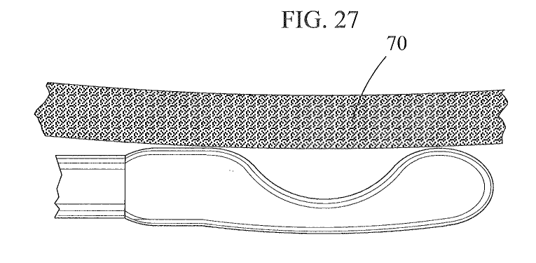

FIG. 18 shows the cross-section, radial arc of the invention. This radial shape covers 206 degrees of the port OD. This arc extends slightly more than two thirds of the internal lumen OD of the tube 11.

FIG. 19 shows the cross-sectional radial arc of Quinn U.S. Pat. No. 5,810,787 that extends 0.186 degrees around the bolus, or slightly below the midpoint or radius of the tube lumen.

FIG. 20 shows the port of expired (Quinn) U.S. Pat. No. 4,594.074 that extends to the OD of the tube's internal OD and forms a port extending 106 degrees around the bolus.

FIG. 21 shows a cross section of a conventional, punched side hole port that has a small cross sectional area and is easily occluded during aspiration.

FIG. 22 illustrates in phantom form the actual shape of the port area 22 of in the bolus invention. FIG. 23 shows the actual effective cross-sectional area of the invention bolus port area 22 in a “flattened” form.

FIG. 24 shows the effective flattened area of Quinn U.S. Pat. No. 5,810,787 FIG. 25 shows the effective flattened area of Quinn U.S. Pat. No. 4,594,674 and

FIG. 26 the punched side hole. Extending the depth of the vertical side of the ports and extending the longitudinal shape of the port increases the overall actual cross-sectional size of the port by approximately 40% over U.S. Pat. No. 5,810,787 which relates to the 41% size increase shown in the vertical side cross-section shown in FIGS. 16 and 17.

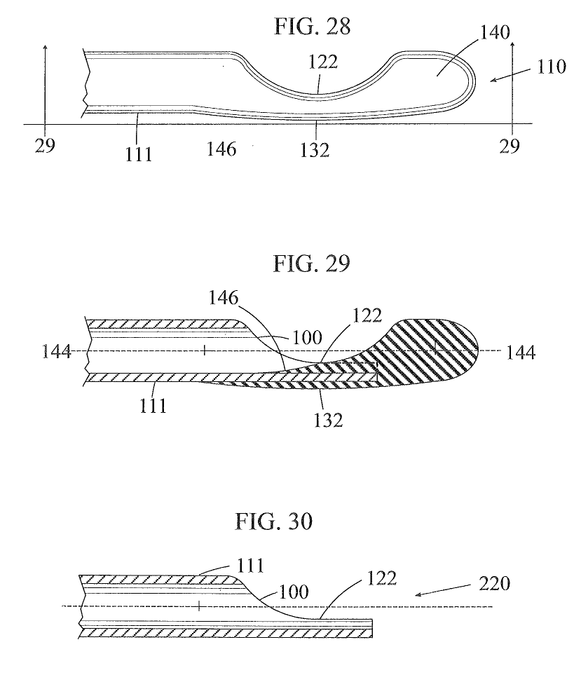

FIG. 28, shows an alternative method of fabricating the bolus tip known as over-molding. In over-molding a conventional cylindrical tube is cut or skived to form part of the port and to also leave a portion of the tube to be a base for over-molding. This over-molding permanently attaches the molded part of the tube to the skived tube. This fabrication method provides a bolus that is the same outside diameter as the tube, which is ideal for catheters that reside in a blood vessel or other restricted space. The attached bolus method described in FIGS. 1 through 26 is ideal for uses where the bolus will ultimately reside in a body cavity such as the stomach because the bolus provides a larger OD and resultant larger and deeper side ports. The elliptical bullet tip and tapered elliptical bolus edges of the new invention minimize any trauma caused by the larger attached bolus during insertion, removal or in-situ.

While preferred embodiments of the invention have been described, it should be noted that the invention is not so limited and modifications may be made without departing from the invention. The scope of the invention is defined by the appended claims, and all devices that come within the meaning of the claims, either literally or by equivalence, are intended to be embraced therein.

I claim:

1. In a catheter comprising a bolus connected to a catheter tube, the bolus port comprising:
a. a tubular shaped body having at an end section at one end, a tip section at the other end and a passage section between said one end section and said tip section, said passage section having a port opening therefrom;
b. a bolus body segment in said passage section opposite said port, said body segment connecting said one end section and said tip section;
c. said body segment forming a side wall in each side of said passage section, each side wall having a portion with a height less than 25% of the ID (internal diameter) of the tube lumen of said body such that said body segment extends around 206 degrees of the circumference of the tube lumen;
d. said body component also including a structural component protruding radially therefrom the same perpendicular radial axis as the aforementioned radial port passage effective to prevent said body segment from bending and restricting said port;
e. said body component also including a curved flow lumen floor segment than rises in a radial fashion from the floor to the lowest point of the passage wall at less than 25% of the tube lumen ID;
f. said curve begins at the point where port lumen first becomes open at the top of its ID, and said rising curve thereby leaves the port completely open for fluid flow.

2. In a catheter having a bolus at an end of a tube section, said tube section containing a lumen of predetermined ID, said bolus comprising:
a. a tubular shaped body having an end section at one end, a tip section at the other end and a passage section between said one end section and said tip section, said passage section having a port opening therefrom;
b. a bolus body segment in said passage section opposite said port, said body segment connecting said one end section and said tip section;
c. said body segment forming a side wall in each side of said passage section, each side wall having a portion with a height less than 75% of an outside radius of said bolus body such that said body segment extends around at least 206% of the OD circumference of said body;
d. said post defining a concave structural arch of a first predetermined length axially in said body segment and forming the periphery of said post; and
e. said body segment also including a convex structural arch of second predetermined length axially in said body segment and protruding outwardly therefrom;
f. said concave structural arch and said convex structural arch cooperating to effectively prevent said body segment from bending said tubular-shaped body and restricting said port.

3. The catheter bolus of claim 2 further comprising:
a. said passage section containing two identical elliptical surface portions that connect a top-most section of the passage of a symmetrical radial passage to the tube connector section and the top section;
b. said passage section also containing a floor that curves upwardly to form a flow direction passage that terminates at the midpoint of the port defining concave arch.

4. The catheter bolus of claim 3 further characterizing in that:
a. said second predetermined length of said convex structural arch is greater than said first predetermined length of said concave structural arch.

5. In a corporeal catheter, comprising:
a. a catheter tube containing a fluid flow passage of predetermined flow capacity;
b. bolus connected to a distal end of said catheter tube;
c. said bolus containing a tubular shaped body having at an end section, a tip section at the other end and a passage section between said one end section and said tip section, said passage section having a port opening therefrom;
d. a bolus body segment in said passage section opposite said port, said body segment connecting said one end section and said tip section;
e. said body segment forming a side wall in each side of said passage section, each side wall having a portion with a height less than 75% of an outside diameter of said body such that said body segment extends around at least 206 degrees of a said circumference of said body;
f. said body segment also including a structural component protruding radially therefrom the same perpendicular radial axis as the aforementioned radial port passage effective to prevent said body segment from bending and restricting said port;
g. said body segment also including a curved flow lumen floor that rises in a radial fashion from the floor to the lowest point of the passage wall at less than 75% of the tube diameter and said floor curve begins at the point where the bolus passage is completely open.
